(19) United States
(12) Patent Application Publication
(10) Pub. No.: US 2006/0075801 A1
(43) Pub. Date: Apr. 13, 2006

(54) Device for preparing a fluid particularly a drilling mud, associated preparation method and associated analysis unit

(76) Inventors: Jean-Francois Evrard, Montreuil (FR); Francois Girardi, Cormeilles en Parisis (FR)

Correspondence Address: WENDEROTH, LIND & PONACK, L.L.P., 2033 K STREET N. W., SUITE 800, WASHINGTON, DC, 20006-1021, US

(21) Appl. No.: 11/245,008

(22) Filed: Oct. 7, 2005

(30) Foreign Application Priority Data
Oct. 11, 2004 (FR) 04 10692

(51) Int. Cl. G01N 1/44; E21B 21/06; E21B 49/08; G01N 33/24; G01N 1/14

(52) U.S. Cl. 73/19.09; 166/302; 166/57; 175/17; 175/217; 73/863.12

(57) ABSTRACT

This device (53) comprises a pump (65) which comprises at least a fixed element (73) and at least a moving element (75) that can move with respect to the fixed element (73). These elements (73, 75) delimit at least one pumping cavity (77). The device (53) also comprises means (151) for heating the mud, these means being applied to the fixed element (73) and/or to the moving element (75) in order to heat said element (75). Application to the analysis of the gaseous content of mud resulting from the drilling of an oil well.

The present invention relates to a device for preparing a fluid, particularly a drilling mud, of the type comprising:
a pump comprising at least a fixed element and at least a moving element that can move with respect to the fixed element, these elements delimiting at least one pumping cavity; and
means for heating the fluid.

This device applies to the pumping and heating of fluids, particularly heat-sensitive fluids, liable to degrade at a temperature close to the temperature to which they are heated, such as drilling muds for example.

When drilling an oil well or some other effluent (particularly gas, vapor, water), it is now practice to analyze the gaseous components contained in the drilling muds emerging from the well. This analysis makes it possible to reconstruct the geological series of formations crossed during drilling and plays a part in determining the possibilities for exploiting the fluid deposits encountered.

This analysis, performed continuously, comprises two main phases. The first phase is to take samples of mud and to extract the gases contained in these samples (for example hydrocarbon compounds, carbon dioxide, hydrogen sulfide). The second phase is to qualify and quantify the gases extracted.

In the first phase, a gas separator with mechanical agitation of the type described in FR 2 799 790 is often used. This type of gas separator comprises a preparation device of the aforementioned type, comprising a pump to convey the mud into a gas extraction vessel. The device further comprises a module for heating the mud, located between the pump and the vessel, to raise the temperature of the mud to values lying, for example, between 70° C. and 120° C. so as to encourage the extraction of gas and increase the sensitivity of the analysis. To do this, the outlet of the pump is connected to the inlet of the heating module by tubing. The heating module comprises, for example, a wall heated indirectly by an electrical resistive element, the wall being immersed in the mud.

However, certain drilling muds are heat-sensitive fluids which degrade at temperatures of around 90° C. In order to raise the mud to the desired temperature value, the electrical resistive element has to be set, in certain instances, to a temperature higher than the temperature at which the mud degrades. Near the heated wall, the mud degrades and soils the wall.

Analysis of the gases has therefore to be periodically interrupted in order to clean the heated wall, and this detracts from the reliability of the measurements taken during drilling. To alleviate this problem, the temperature to which the mud is heated is reduced, and this lessens the sensitivity of the analysis.

It is the main object of the invention to increase the reliability of the analysis of the gases contained in a mud during drilling, without impairing its sensitivity.

To this end, the subject of the invention is a device of the aforementioned type, characterized in that the heating means are applied to the fixed element and/or to the moving element in order to heat said element.

The device according to the invention may comprise one or more of the following characteristics, taken in isolation or in any technically feasible combination:

the pumping cavity is delimited by at least two locally contacting moving surfaces, the displacement of the moving element with respect to the fixed element causing relative displacement of said locally contacting surfaces one with respect to the other, so as to scrape said surfaces;

of said fixed element and moving element, one is a female element delimiting a cavity, and the other is a male element located in the female element;

the heating means are located in the male element;

the heating means are located in the female element;

it comprises a thermal insulation jacket located around the female element;

the female element exhibits an internal surface of substantially helical shape, the male element exhibiting an external surface of substantially helical shape, said internal and external surfaces delimiting a plurality of non-contiguous pumping cavities;

the moving element is mounted to rotate about an axis of rotation and it comprises means for the rotational drive of the moving element with respect to the fixed element about the axis of rotation;

the heating means are located in the moving element and are electrically connected to a source of electrical power through the rotational drive means.

A further subject of the invention is a method for preparing a fluid, particularly a drilling mud, of the type comprising the steps of:

pumping the fluid between an inlet of a pump and an outlet of the pump, the pump comprising at least a fixed element and at least a moving element that can move with respect to the fixed element, these elements delimiting at least one pumping cavity; and

heating the fluid using heating means; characterized in that the heating of the fluid is performed by contact of this fluid with a surface of the fixed element and/or of the moving element of the pump, this surface being heated by the heating means.

Another subject of the invention is a unit for analyzing at least one gas contained in a fluid, particularly a drilling mud, flowing through a line, characterized in that it comprises:

means for sampling the fluid in said line;

a device as defined hereinabove, an inlet of the pump being connected to the sampling means;

means for extracting the gas contained in the fluid, these means being connected to an outlet of the pump; and

means for analyzing the gases extracted.

An exemplary embodiment of the invention will now be described with reference to the attached drawings, in which:

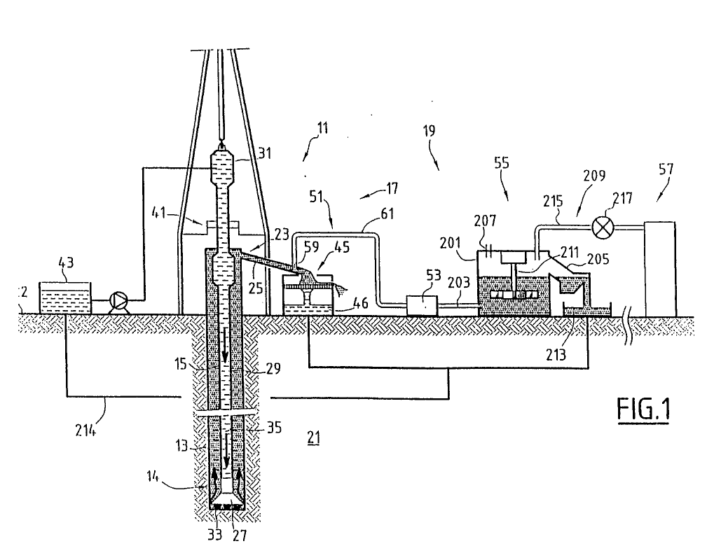

FIG. 1 is a schematic view in vertical section of a drilling installation, equipped with an analysis unit according to the invention;

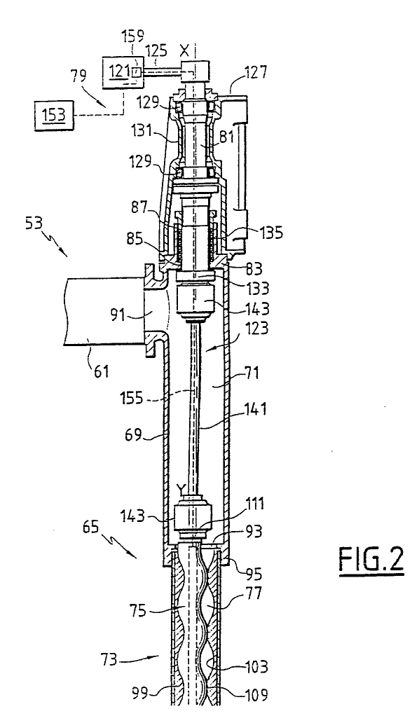

FIG. 2 is a schematic view in section on a horizontal plane of a first preparation device according to the invention; and

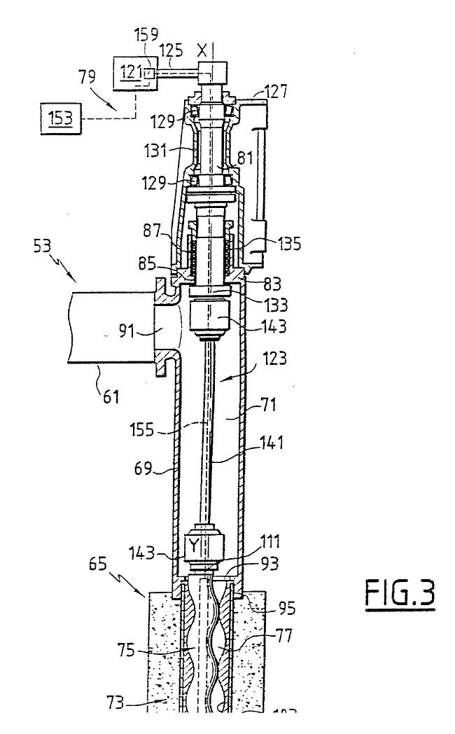

FIG. 3 is a view similar to FIG. 2 of a second preparation device according to the invention.

A base analysis unit according to the invention is used for example in a drilling installation 11 for drilling an oil well.

As illustrated by FIG. 1, this installation 11 comprises a drilling line 13 in a cavity pierced by a rotary drilling tool 15, a surface installation 17 and a gas analysis unit 19 according to the invention.

The drilling line 13 is positioned in the cavity drilled in the subsoil 21 by the rotary drilling tool 15. This line 13 comprises, at the surface 22, a wellhead 23 equipped with a drain pipe 25.

The drilling tool 15 comprises a drilling head 27, a drill string 29, and a head 31 for injecting liquid.

The drilling head 27 comprises means 33 for drilling through rocks in the subsoil 21. It is mounted at the bottom of the drill string 29 and positioned in the bottom of the drilling line 13.

The string 29 comprises a collection of hollow drilling tubes. These tubes delimit an internal space 35 allowing liquid to be conveyed from the surface 22 to the drilling head 27. For this, the head 31 for injecting liquid is screwed onto the top of the string 29.

The surface installation 17 comprises means 41 for supporting and rotationally driving the drilling tool 15, means 43 for injecting drilling liquid and a vibrating screen 45.

The injection means 43 are hydraulically connected to the injection head 31 in order to introduce and circulate a liquid in the internal space 35 of the drilling string 29.

The vibrating screen 45 collects, in a liquid collecting vessel 46, the liquid laden with drilling residue, hereinafter termed “drilling mud” that leaves the drain pipe 25 and separates the liquid from the solid drilling residue.

As illustrated in FIG. 1, the analysis unit 19 comprises mud sampling means 51 tapped into the drain pipe 25, a device 53 for preparing the mud, means 55 for extracting the gases contained in the mud, and means 57 for analyzing the gases extracted.

The sampling means 51 comprise a liquid sampling head 59 mounted to project into the drain pipe 25, and tubing 61 connecting the head 59 to the preparation device 53.

As variant, the sampling means are tapped into a vessel that collects the liquid into which vessel the drain pipe 25 opens, for example the vessel 46.

As illustrated by FIG. 2, the preparation device 53 comprises a Moineau pump 65 equipped with means 67 for heating the mud.

The pump 65 comprises a casing 69 which delimits a mud inlet chamber 71, a stator 73 and a rotor 75 which delimit a plurality of unconnected pumping cavities 77 and means 79 for the rotational drive of the rotor 75 with respect to the stator 73 and comprising a driveshaft 81.

The casing 69 has a tubular overall shape running along the longitudinal axis X-X′ of the pump 65.

In everything that follows, the terms “upstream” and “downstream” are to be understood as meaning with respect to the direction in which the fluid normally flows in the installation.

At its upstream end 83 the casing 69 has an axial opening 85 to accept the driveshaft 81, this opening being delimited by an internal annular flange externally extended by an annular support sleeve 87.

The inlet chamber 71 extends between a lateral inlet opening 91 formed near the upstream end 83 and an axial pumping opening 93 formed at the downstream end 95 of the casing 69. The inlet opening 91 is connected to the connecting tubing 61.

The stator 73 is mounted in the axial continuation of the casing 69 at the downstream end 95 and comprises an outer jacket 97 and internal packing 99.

The outer jacket 97 is fixed to the casing 69 in the continuation of the pumping opening 93. At its free end it has a delivery passage 101 of the pump 65.

The packing 99 is fixed to an internal wall of the jacket 97. It has an internal surface of helical shape running between the pumping opening 93 and the delivery passage 101.

In the example illustrated in FIG. 2, the internal helical surface 103 is generated in a double helix with five revolutions.

The packing 99 is based on a heat-resistant and thermally-insulating elastomeric material such as nitrile or Viton® for example.

Thus, heat losses in the pump 65 are minimized and the heating efficiency of the pump 65 is improved.

The rotor 75 comprises a rotary metal bar 105 mounted in the internal cavity delimited by the packing 99 of the stator 73.

The bar 105 exhibits a helical external surface 107. The external surface 107 of the bar 105 is generated for example by a single helix, the dimensions of which are chosen so that the internal surface 103 of the packing 99 and the external surface 107 of the bar 105 possess a plurality of regions 109 of contact. These contact regions 109 delimit the plurality of cellular pumping cavities 77, sealed with respect to one another.

The bar 105 is, for example, based on a metal of the stainless steel type, or a steel with a surface treatment of nickel plating, or bronze, the thermal conductivity of which lies between 15 W/m²·K and 200 W/m²·K. The length of the bar is between 0.2 m and 0.8 m and its external surface area 107 ranges between 5×10⁻³ m² and 1×10⁻¹ m² so as to raise a mud flowing at a flow rate of between 5 l/h and 150 l/h to temperatures of between 20° C. and 300° C.

The bar 105 is mounted to rotate in the packing 99 about the longitudinal axis X-X′ of the pump 65. Given the relative configuration of the internal and external helical surfaces 103, 107, the central axis Y-Y′ of the bar 105 is laterally offset from the longitudinal axis X-X′ and parallel to this axis X-X′.

Thus, when the bar 105 is rotationally driven about the longitudinal axis X-X′, its upstream end 111 describes a circular movement about this axis X-X′. Furthermore, as the bar 105 rotates about the longitudinal axis X-X′, the contact regions 109 travel in the downstream direction and the cellular cavities 77 travel longitudinally from the pumping opening 93 towards the delivery passage 101 of the pump 65.

During this displacement, the contact regions 109 are swept over the entire internal surface 103 of the packing 99 and the entire external surface 107 of the bar 105, to allow the internal and external surfaces 103 and 107 to be scraped continuously so that any residue deposited on these surfaces 103 and 107 is removed to the delivery passage 101.

The means 79 for the rotational drive of the rotor 75 comprise, apart from the driveshaft 81 of the rotor 75, an electric motor 121 and transmission means 123 providing transmission between the driveshaft 81 and the rotor 75.

The motor 121 is located outside the casing 69. It comprises an output shaft 125 enmeshed with the driveshaft 81 to allow the latter to be turned.

The driveshaft 81 is mounted to rotate about the longitudinal axis X-X′ in a hollow box 127 fixed in the axial continuation of the upstream end 83 of the casing 69. Upstream rolling bearings 129 are positioned between a restriction 131 of the box 127 and the shaft 81.

The downstream end 133 of the shaft 81 is also mounted to rotate in the support sleeve 87 of the casing 69 via a downstream rolling bearing 135. The downstream end 133 projects into the inlet chamber 71 through the accepting opening 85. An annular seal (not depicted) seals between the annular flange and the shaft 81.

The transmission means 123 comprise a connecting rod 141 articulated, on the one hand, to the downstream end 133 of the driveshaft 81 and, on the other hand, to the upstream end 111 of the bar 105.

The articulations 143 between the connecting rod 141 and the driveshaft 81, on the one hand, and the connecting rod 141 and the bar 105 on the other hand, have at least two degrees of freedom about two perpendicular axes. These articulations 143 are, for example, of the cardan or ball joint type, so as to allow the upstream end 111 of the bar 105 to describe a more or less circular movement in a plane perpendicular to the longitudinal axis X-X′.

The heating means 67 comprise an electrical resistive element 151, a source of electrical power 153 for this resistive element 151 and means 155 of electrical connection between this source 153 and the resistive element 151.

The electrical resistive element 151 comprises a heating cartridge of more or less cylindrical shape located in a cavity 157 of corresponding shape, formed in the bar 105. As a preference, the cartridge has an external surface area of between 2.10⁻² m² and 4.10⁻² m² and a thermal power of between 1000 W and 4000 W.

The source 153 of electrical power is positioned outside the casing 69, near the motor 121.

The electrical connecting means 155 comprise electrical pathways running between the resistive element 151 and a rotary commutator 159 of the brush type positioned between the output shaft 125 and the motor 121. The pathways run in succession through the rotor 75, the connecting rod 141, the driveshaft 81 and the output shaft 125.

The rotary commutator 159 is positioned some distance from the heating resistive element 151, which limits the extent to which it heats up and thus ensures that it will operate reliably.

With reference to FIG. 1, the extraction means 55 are, for example, analogous to those described in FR 2 799 790. They comprise an enclosure 201, a pipe 203 for conveying mud to the enclosure 201, a pipe 205 for removing mud from the enclosure 201, means 207 for introducing a carrier gas into the enclosure 201, and a pipe 209 for extracting the extracted gases from the enclosure 201.

The enclosure 201 is fitted with agitating means 211.

The mud conveying pipe 203 runs between the delivery passage 101 of the pump 65 and an inlet opening formed in the lower part of the enclosure 201.

The discharge pipe 205 extends between an overspill passage formed in an upper part of the enclosure 201 and a holding tank 213 intended to receive the mud discharged from the enclosure 201. As an alternative, the tank 213 is formed by the collecting vessel 46 that contains the vibrating screen 45.

The mud collected in the holding tank 213 and in the vessel 46 is recirculated to the injection means 43 by a mud recirculation pipe 214.

The means 207 for introducing a carrier gas into the enclosure 201 comprise an air or neutral-gas tapping opening into the enclosure 201.

The extraction pipe 209 runs between an extraction opening formed in the upper part of the enclosure 201 and the analysis means 57. It comprises a transport line 215 and suction means 217.

The analysis means 57 comprise instrumentation for detecting and quantifying one or several gases extracted.

The way in which the analysis unit according to the invention works when drilling a well will now be described by way of example.

During a drilling phase, the drilling tool 15 is rotationally driven by the surface installation 41. A drilling liquid is introduced into the interior space 35 of the drill string 29 via the injection means 43. This liquid descends as far as the drilling head 27, and passes into the drilling line 13 through the drilling head 27. This liquid cools and lubricates the drilling means 33. Next, the liquid collects the solid spoil resulting from the drilling and rises up through the annular space between the drill string 29 and the walls of the drilling line 13, and is then removed through the drain pipe.

With reference to FIG. 2, the motor 121 is then activated to rotationally drive the driveshaft 81 about the longitudinal axis X-X′. The rotational movement of the driveshaft 81 is transmitted to the rotor 75 via the transmission means 123.

The relative displacement of the rotor 75 with respect to the stator 73 causes the contact regions 109 to move and as a result causes the cellular cavities 77 to travel from the downstream opening 93 towards the delivery passage 101.

The source of electrical power 153 is activated. This source 153 powers the electrical resistive element 151 via the connecting means 155. The resistive element 151 is raised to a temperature of between 50° C. and 300° C. at its surface.

The pump is self-priming which means that the mud is continuously taken from the drain pipe 25 or from the vessel 46 by the sampling means 51, then conveyed by suction through the sampling tubing 61 as far as the inlet chamber 71. The mud then enters a cavity 77 adjacent to the pumping opening 93 and is then pumped by the downstream displacement of this cavity 77 as far as the delivery passage 101.

As the mud passes through the cavity 77, the heat emitted by the electrical resistive element 151 is transmitted to the mud by conduction through the metal bar 105 then by convection at the external surface 107. The temperature at the external surface of the bar is, for example, between 50° C. and 300° C.

The mud is thus raised to the desired temperature, for example of between 70° C. and 120° C., as it passes through the pump 65.

By virtue of the large surface area for exchange between the mud and the rotor 75, the temperature difference between the external surface 107 of the rotor 75 and the mud is minimized, making it possible to appreciably reduce the extent to which this surface 107 becomes soiled.

The mud is then conveyed as far as the enclosure 201 through the inlet pipe 203. The agitating means 211 are activated in order to extract the gases contained in the mud.

The extracted gases are sampled by the pipe 209 and conveyed by suction as far as the analysis means 57 where they are qualified and quantified.

In the analysis unit 19 according to the invention, there is no longer any need to plan periodic cleaning of the heating means 67 during drilling, and this considerably improves the reliability of the analysis of the gases contained in the drilling mud.

As an alternative, a thermal regulating device (not depicted) regulates the electrical power delivered to the electrical resistive element 151 in order to control the mud outlet temperature, taken at the delivery passage 101 or in the enclosure 201.

In a second alternative form illustrated by FIG. 3, a thermal insulating jacket 301 is arranged around the stator 73, to reduce thermal losses within the pump 65 and thus improve the heating efficiency of this pump 65.

In a third alternative form which has not been depicted, the helical bar 105 is fixed with respect to the casing 69 and the assembly 73 consisting of the packing 99 and the jacket 97 is rotated about the longitudinal axis X-X′. This assembly 73 in this case is provided with rotational drive means.

In this alternative form, the electrical connecting means 155 do not need to have a rotary commutator 159 because the bar 105 is fixed.

In a fourth alternative form, the maximum transverse dimension of the pumping cavities is increased in order to accept rocky debris of larger size.

In a fifth alternative form, the electrical resistive element 151 is located in the stator 73.

By virtue of the invention which has just been described, it is possible to have a device for preparing a drilling mud, with a view to extracting the gases from this mud, which raises the mud to a determined temperature while at the same time limiting the extent of soiling of the device.

The reliability of the analysis of the gases contained in a drilling mud extracted from an installation equipped with an analysis unit according to the invention is therefore appreciably improved by this device.

Furthermore, this device makes it possible to increase the temperature to which the mud is raised before the gases are extracted without causing substantial soiling of the device, thanks to the continuous scraping of the pumping cavities. The extraction efficiency for the extraction of the gases contained in the mud is therefore improved and the sensitivity of the analysis is increased.

This device also limits the space occupied by the analysis unit positioned in an explosive area near the well.

The device can also be adapted to suit other types of pumps, for example piston pumps.

More generally, the device applies to the pumping and simultaneous heating of other heat-sensitive fluids such as milk for example.

1. Device (53) for preparing a fluid, particularly a drilling mud, of the type comprising:
a pump (65) comprising at least a fixed element (73) and at least a moving element (75) that can move with respect to the fixed element (73), these elements (73, 75) delimiting at least one pumping cavity (77); and
means (151) for heating the fluid;
characterized in that the heating means (151) are applied to the fixed element (73) and/or to the moving element (75) in order to heat said element (75).

2. Device (53) according to claim 1, characterized in that the pumping cavity (77) is delimited by at least two locally contacting moving surfaces (103, 107), the displacement of the moving element (75) with respect to the fixed element (73) causing relative displacement of said locally contacting surfaces (103, 107) one with respect to the other, so as to scrape said surfaces (103, 107).

3. Device (53) according to claim 1, characterized in that of said fixed element (73) and moving element (75), one is a female element (73) delimiting a cavity, and the other is a male element (75) located in the female element (73).

4. Device (53) according to claim 3, characterized in that the heating means (151) are located in the male element (75).

5. Device (53) according to claim 3, characterized in that the heating means (151) are located in the female element (73).

6. Device (53) according to claim 3, characterized in that it comprises a thermal insulation jacket (301) located around the female element (73).

7. Device (53) according to claim 3, characterized in that the female element (73) exhibits an internal surface (103) of substantially helical shape, the male element (75) exhibiting an external surface (107) of substantially helical shape, said internal (103) and external (107) surfaces delimiting a plurality of non-contiguous pumping cavities (77).

8. Device (53) according to claim 1, characterized in that the moving element (75) is mounted to rotate about an axis (X-X′) of rotation and in that it comprises means (79) for the rotational drive of the moving element (75) with respect to the fixed element (73) about the axis of rotation (X-X′).

9. Device (53) according to claim 8, characterized in that the heating means (151) are located in the moving element (75) and are electrically connected to a source of electrical power (153) through the rotational drive means (121).

10. Method for preparing a fluid, particularly a drilling mud, of the type comprising the steps of:
pumping the fluid between an inlet (91) of a pump (65) and an outlet (101) of the pump (65), the pump (65) comprising at least a fixed element (73) and at least a moving element (75) that can move with respect to the fixed element (73), these elements (73, 75) delimiting at least one pumping cavity (77); and
heating the fluid using heating means (151);
characterized in that the heating of the fluid is performed by contact of this fluid with a surface (107) of the fixed element (73) and/or of the moving element (75) of the pump (65), this surface (107) being heated by the heating means (151).

11. Unit (51, 53, 55, 57) for analyzing at least one gas contained in a fluid, particularly a drilling mud, flowing through a line (25), characterized in that it comprises:
means (51) for sampling the fluid in said line (25);
a device (53) according to claim 1, an inlet (91) of the pump (65) being connected to the sampling means (51);
means (55) for extracting the gas contained in the fluid, these means being connected to an outlet (101) of the pump (65); and
means (57) for analyzing the gases extracted.

12. Device (53) according to claim 2, characterized in that of said fixed element (73) and moving element (75), one is a female element (73) delimiting a cavity, and the other is a male element (75) located in the female element (73).

13. Device (53) according to claim 12, characterized in that the heating means (151) are located in the male element (75).

14. Device (53) according to claim 12, characterized in that the heating means (151) are located in the female element (73).

15. Device (53) according to claim 12, characterized in that it comprises a thermal insulation jacket (301) located around the female element (73).

16. Device (53) according to claim 4, characterized in that it comprises a thermal insulation jacket (301) located around the female element (73).

17. Device (53) according to claim 13, characterized in that it comprises a thermal insulation jacket (301) located around the female element (73).

18. Device (53) according to claim 5, characterized in that it comprises a thermal insulation jacket (301) located around the female element (73).

19. Device (53) according to claim 14, characterized in that it comprises a thermal insulation jacket (301) located around the female element (73).

20. Device (53) according to claim 12, characterized in that the female element (73) exhibits an internal surface (103) of substantially helical shape, the male element (75) exhibiting an external surface (107) of substantially helical shape, said internal (103) and external (107) surfaces delimiting a plurality of non-contiguous pumping cavities (77).
